(19) United States
(12) Patent Application Publication
(10) Pub. No.: US 2024/0316308 A1
(43) Pub. Date: Sep. 26, 2024

(54) Sensory Enclosure Assembly and Method of Improving Sensory Related Conditions

(71) Applicant: zPods Holdings LLC, St. Peters, MO (US)

(72) Inventors: Gary KELLMANN, St. Peters, MO (US); Jonas BUXTON, St. Charles, MO (US); Charles GRANT, Staples, MN (US); Gregory Koeller, Apple Valley, MN (US)

(21) Appl. No.: 18/604,103

(22) Filed: Mar. 13, 2024

Related U.S. Application Data
(60) Provisional application No. 63/454,696, filed on Mar. 26, 2023.

(51) Int. Cl. A61M 21/00; A61M 21/02

(52) U.S. Cl. CPC A61M 21/0094; A61M 21/02; A61M 2021/0016; A61M 2021/0022; A61M 2021/0027; A61M 2021/005

(57) ABSTRACT

A sensory enclosure system for improving the sensory management or sleeping habits of individuals with sensory sensitivities or sleeping problems. The system has an interior formed by multiple wall panels that connect to or mate with a platform intended to accommodate furniture such as chairs, mattresses, and bean bags. The enclosure's interior is enclosed by a ceiling or roof panel that connects to or mates with the wall panels. Additionally, the system features one or more door panels to allow entry and exit to the interior space. It may also incorporate an AI-environmental controller capable of managing various environmental factors and communicating with remote devices that are monitoring environmental factors and a user of the system.

### PRIORITY

This application claims the benefit of U.S. Provisional Patent Application No. 63/454,696 filed on Mar. 26, 2023.

### FIELD

The present invention relates generally to sensory enclosures and methods of improving the sensory related conditions, and, more particularly, to a sensory enclosure assembly and methods of improving the sensory conditions of individuals of all ages, groups, and neurotypes who may feel dysregulated in any way by their surrounding environment.

### BACKGROUND

It is estimated that 5% to 16.5% of the general population have symptoms associated with one or more sensory conditions. Sensory conditions can impact various facets of an individual's life. For example, 50 to 70 million Americans have chronic sleep problems, many of which may have challenges with sensory conditions. Further, as many as 50% of kids will experience a sleep problem during their childhood. Chronic sleep problems usher in a host of health problems that can further exacerbate sleep problems. For instance, it is well known that people who sleep less than 7 hours per night are more likely to develop obesity than those who sleep more. Obesity can lead to sleep apnea, which further causes sleep deprivation.

Other risks associated with sleep problems include the development of diabetes and hypertension. An individual's risk of developing diabetes increases with too little sleep (less than 7 hours) and too much sleep (more than 9 hours). Individuals that sleep less than six hours per night are considered to be 20% to 32% more likely to develop hypertension than people who get 7 to 8 hours of sleep per night.

One group of individuals that often have sensory related conditions is neurodivergent individuals. Neurodiversity is a term used to describe a variation in the experience that individuals encounter during their daily lives (e.g., school, work, and social relationships). It is estimated that 15-20 percent of the world's population exhibits some form of neurodivergence. neurodivergent individuals include individuals with autism, ADHD, PTSD, and OCD, to name a few.

Neurodivergent individual's families and their caregivers often have to deal with the unique sleeping patterns and habits experienced by neurodivergent individuals. The unique sleeping patterns and habits may include difficulty falling asleep, trouble settling down and relaxing before bedtime, difficulty staying asleep, and waking up frequently during the night. Having one or more of the preceding sleeping patterns or habits typically results in the neurodivergent individual, their family, and their caregiver, unable to get a full night's rest.

Individuals with chronic sleep problems are often resistant to bedtime routines. They often engage in active resistance to attempts by their caregivers to alter their current sleeping pattern or bedtime routine. The active resistant behaviors can include repeatedly brushing their teeth, changing into different pajamas, roaming the house, playing video games, or watching movies/videos rather than falling asleep.

Neurodivergent individuals, especially children with autism spectrum disorder (“ASD”), often have sensory sensitivities, such as being sensitive to the feeling of certain bedding or being overly sensitive to light or noise, all of which can impact their sleep and overall well-being. Their sensory sensitivities compel them to engage in “escape space” behaviors where they seek enclosures that make them feel safe. For instance, some individuals will seek enclosures such as the space beneath their beds, their closets, and in some instances, even a bathtub or shower.

Understanding sensory sensitivities and their impacts on individuals is crucial for parents, spouses, families, and caregivers. Implementing strategies that address sensory sensitivities can significantly improve the well-being of those affected. Among these strategies are the following methods to enhance or improve sleeping habits:
Creating a space where they feel safe and comfortable.
Establishing a consistent bedtime routine: Consistency and routine can help increase an individual's comfort level and overall feeling of security at bedtime.
Creating a sensory-friendly environment: This can include reducing noise, light, and other sensory inputs that might interfere with sleep.
Implementing relaxation techniques: This can include deep breathing, reading, or listening to calming music before an activity such as bedtime.
Encouraging physical activity: Regular physical activity during the day can help with sensory sensitivity and help promote a better sleep experience at night.
Using positive reinforcement: Rewarding positive behaviors, such as seeking out a designated sensory controlled environment to help manage stress and anxiety, which will also reinforce good sleep habits.

What is needed is a sensory-friendly environment that mitigates or reduces noise, light, and other sensory stimuli/inputs that could adversely affect a user's well-being, thus fostering healthy sleep habits and routines. What is also needed is a sensory-friendly environment that facilitates the implementation and practice of relaxation techniques, like deep breathing, reading, or listening to calming music. Still another need is a sensory-friendly environment that promotes a sense of comfort, safety, and security, enabling individual/users to alleviate anxiety and nervousness, thereby improving repetitive movement patterns/habits and cause a better night's sleep.

Still another need is a customizable sensory-friendly environment that can be tailored to every individual's unique needs and circumstances.

Still yet another need is to create a sensory-friendly environment that can be selectively enclosed, self-contained, dynamically responsive, and completely controllable by the neurodivergent individual and/or their caregiver. Such an environment positively influences the biochemistry, behavior, and surroundings of individuals with sensory sensitivities. The ability to effectively regulate an individual's biochemistry, behavior, and surroundings is vital for enhancing their overall well-being, including improving their sleep habits.

Yet, another need is a novel sensory enclosure assembly capable of controlling or influencing environmental factors. A system that integrates artificial intelligence (“AI”) technology that is configured to change the sensory enclosure's environment to the individual's sensory needs, including during a bedtime routine and while they are sleeping. There is also a need for an AI-equipped sensory enclosure that is equipped to automatically adjust the environmental factors of the enclosure, enhancing the individual's well-being and facilitating restful sleep, thus minimizing the risk of orthosomnia (obsessive pursuit of sleep through sleep tracker data). What is also needed is an AI-equipped enclosure where the AI serves as a gateway to AI-mediated sensory and sleep behavior therapy, as well as preventative health care measures. There is also a need for an AI-equipped sensory enclosure that is able to communicate with one or more devices or paired devices (e.g., thermostats, mobile phones, healthcare monitoring and control devices (e.g., heartrate monitors, sleep state monitors, blood pressure monitors, positive airflow devices, etc.)) for the purpose of controlling the environmental factors and/or paired devices based upon data shared between the paired devices and the AI.

The above is not intended to limit the scope of the invention, or describe each embodiment, aspect, implementation, feature, or advantage of the invention. The detailed technology and preferred embodiments for the subject invention are described in the following paragraphs accompanying the appended drawings for people skilled in this field to well appreciate the features of the claimed invention. It is understood that the features mentioned hereinbefore and those to be commented on hereinafter may be used not only in the specified combinations, but also in other combinations or in isolation, without departing from the scope of the present invention.

### SUMMARY OF THE INVENTION

The present invention provides a self-contained sensory controllable environment designed to provide a comfortable and relaxing sensory experience. The sensory controllable enclosure or pod is manufactured using rotomolding, a process that creates a seamless and durable structure that can be easily assembled, disassembled, and transported. While there are various configurations possible, the basic or base sensory enclosure is composed of four walls or panels, a ceiling or roof panel, and a support platform or floor, which can be easily assembled and disassembled.

The sensory enclosure features a slidable door, which provides easy access to the interior of the enclosure. The slidable door includes a breakaway feature that allows a user or sleeper to separate the slidable door from the sensory enclosure by applying a force (e.g., kicking) against or onto the slidable door.

Once an individual or user is inside the sensory enclosure, the user can enjoy the benefits of the interior mirror, which includes making the enclosure feel larger, scattering or reflecting calming lights inside the enclosure, and allowing an individual or user to see themselves or reorient themselves when they are dysregulated. A sound system, which is integrated into the sensory enclosure, allows the user to listen to music or other audio and video content. Additionally, the sensory enclosure is equipped with a fan, which provides air circulation, and lights that can be adjusted to create the desired ambiance.

The sensory enclosures of the present invention can also include a mattress or other bedding designed to provide a comfortable sleeping surface and experience. The mattress or bedding is ideally made of high-quality materials and is designed to provide ample support and comfort.

Finally, as briefly mentioned above, multiple sensory enclosures can be stacked on top of one another, providing a convenient and space-saving solution for those who need to provide sensory or sleeping arrangements for multiple users. The design of the sensory enclosure's of the present invention facilitates the easy stacking and securing together of multiple sensory enclosures ensuring that they remain stable and secure. The sensory enclosures of the present invention may also include one or more ladders that can be connectable to one or more of the four walls or panels of the sensory enclosures to allow a user to easily access an upper surface or upper sensory enclosure.

In conclusion, the present invention provides a sensory enclosure that creates a versatile and convenient sensory and/or sleeping controllable solution providing a comfortable and relaxing environment. Whether used as a single unit or stacked to accommodate multiple users, the sensory enclosure is rotomolded from high-quality and durable materials allowing it to meet the needs of a wide range of users.

### BRIEF DESCRIPTION OF THE DRAWINGS

In the accompanying drawings:

FIG. 1 is a perspective view of a sensory enclosure with an optional enclosure or pod rail, in accordance with the invention.

FIG. 2 is an exploded view of the sensory enclosure of FIG. 1 without the ladder.

FIG. 3 is an exploded view of FIG. 1 showing walling panels and support platforms.

FIG. 4 is a perspective view of a wall panel having speakers and an entertainment system.

FIG. 5 is a perspective view of door panels in accordance with the invention.

FIG. 6A is a perspective view of a support surface of a support platform.

FIG. 6B is a perspective view of a bottom surface of a support platform.

FIG. 7A is a cross-sectional view of a ceiling panel mated with wall panels and door panels.

FIG. 7B is a cross-sectional view of a secondary support panel mated with wall panels and door panels.

FIG. 7C is a cross-sectional view of a support platform mated with wall panels and door panels.

FIG. 8A is a perspective view of wall panel channels and door panel channels in accordance with the invention.

FIG. 8B is a perspective view of a door panel showing upper and lower rails in accordance with the invention.

FIG. 8C is a perspective view of the upper rail of FIG. 8B.

FIG. 8D is a perspective view of the lower rail of FIG. 8B.

FIG. 9A is a perspective view of a stackable sensory enclosure system in accordance with the invention.

FIG. 9B is an exploded view of the stackable sensory enclosure system of FIG. 9A.

FIG. 10 is a side view of the stackable sensory enclosure system of FIG. 9A.

FIG. 11 is a side view of the stackable sensory enclosure system of FIG. 9A.

FIG. 12A is a back view of the stackable sensory enclosure system of FIG. 9A without a pod rail.

FIG. 12B is a back view of the stackable sensory enclosure system of FIG. 9A with a pod rail.

FIG. 13 is a cross-sectional view of the stackable sensory enclosure system of FIG. 9A showing an entertainment wall panel.

FIG. 14 is a cross-sectional view of the stackable sensory enclosure system of FIG. 9A showing door panels and support member.

FIG. 15 is a front view of a support member in accordance with the invention.

FIGS. 16A and 16B are perspective views of a ladder in accordance with the invention.

FIG. 17A is a top view of a sensory enclosure in accordance with the invention.

FIG. 17B is a bottom view of a sensory enclosure in accordance with the invention.

While the invention is amenable to various modifications and alternative forms, specifics thereof have been shown by way of example in the drawings and will be described in detail. It should be understood, however, that the intention is not to limit the invention to the particular example embodiments described. On the contrary, the invention is to cover all modifications, equivalents, and alternatives falling within the scope of the invention as defined by the appended claims.

### DETAILED DESCRIPTION

In the detailed description that follows, the invention of the present invention will be described using terminology that is being provided for reference only. The terminology used is not intended to be limiting. The terminology includes but is not limited to “up” or “down”, which may refer to a direction in the drawings. Terminology such as “upper”, “lower”, “top”, “bottom”, “side”, “end”, “horizontal”, and “vertical” are used generally to describe the orientation of parts of the sensory enclosure or pod. These and the other terminology used in this application, including their alternatives, will be apparent to those of ordinary skill in the art of the invention and is not to be limited.

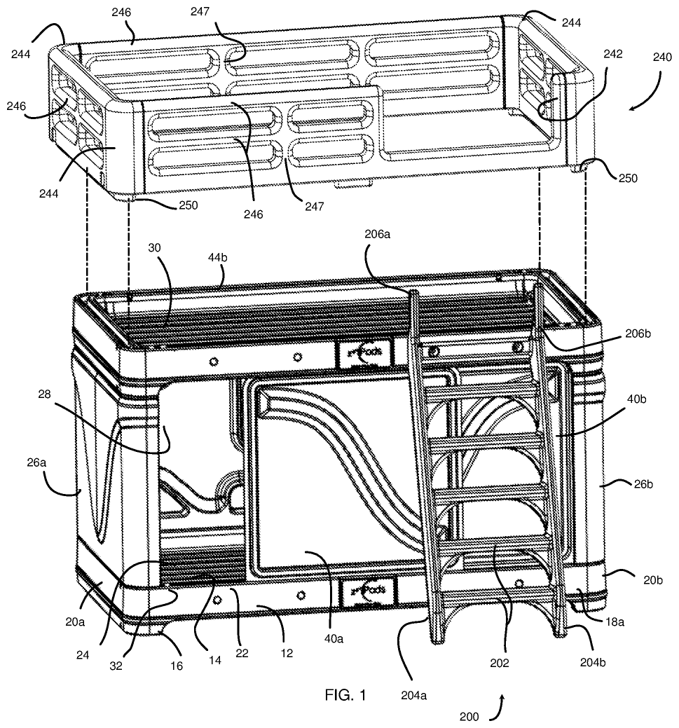

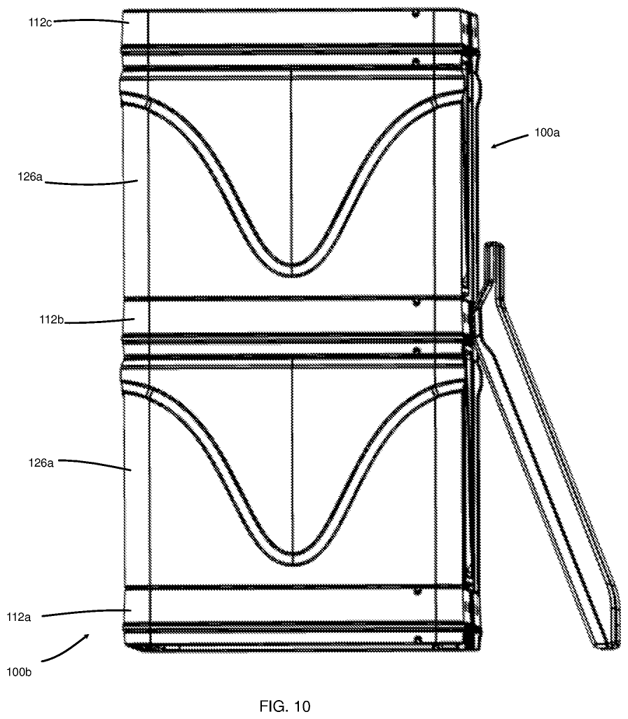

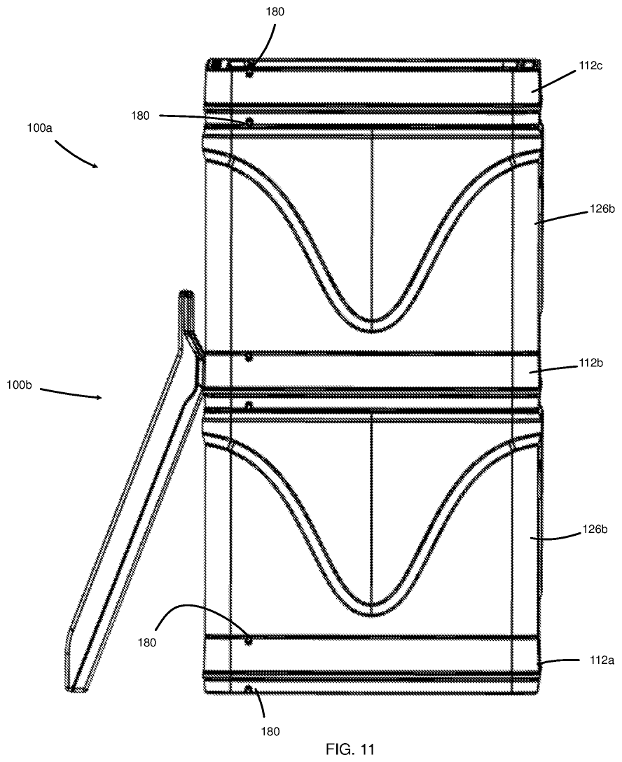

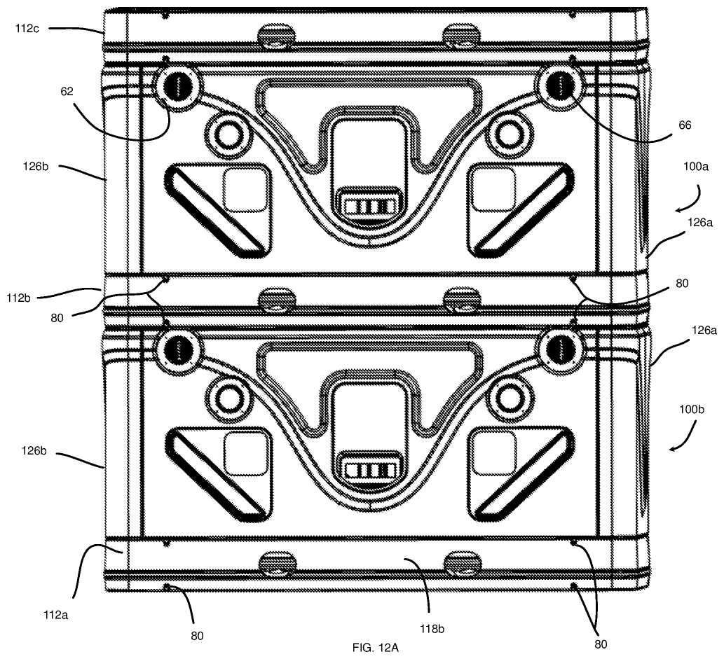

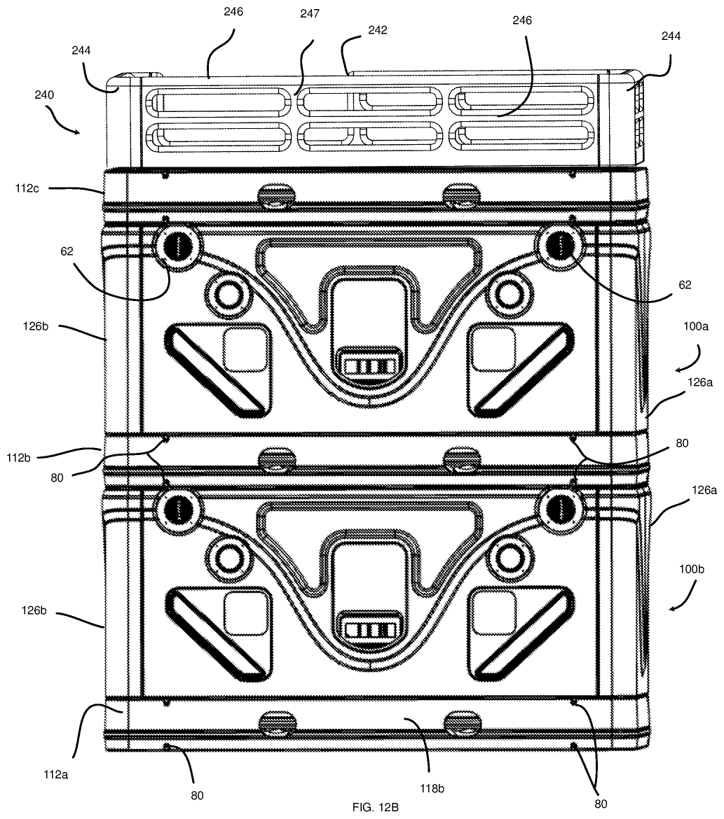

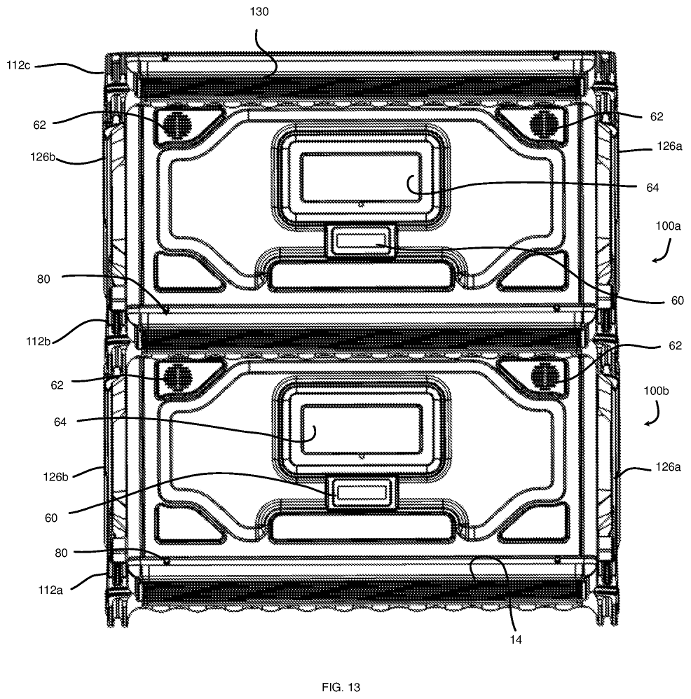

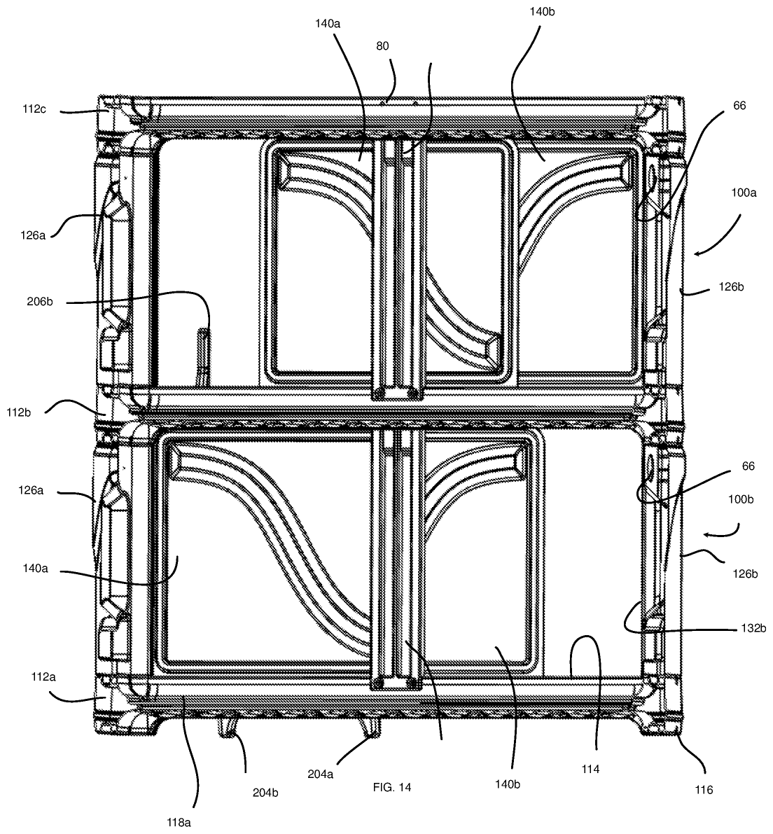

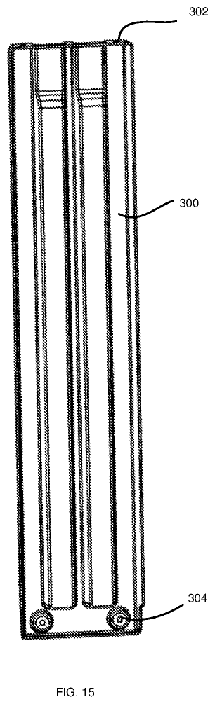

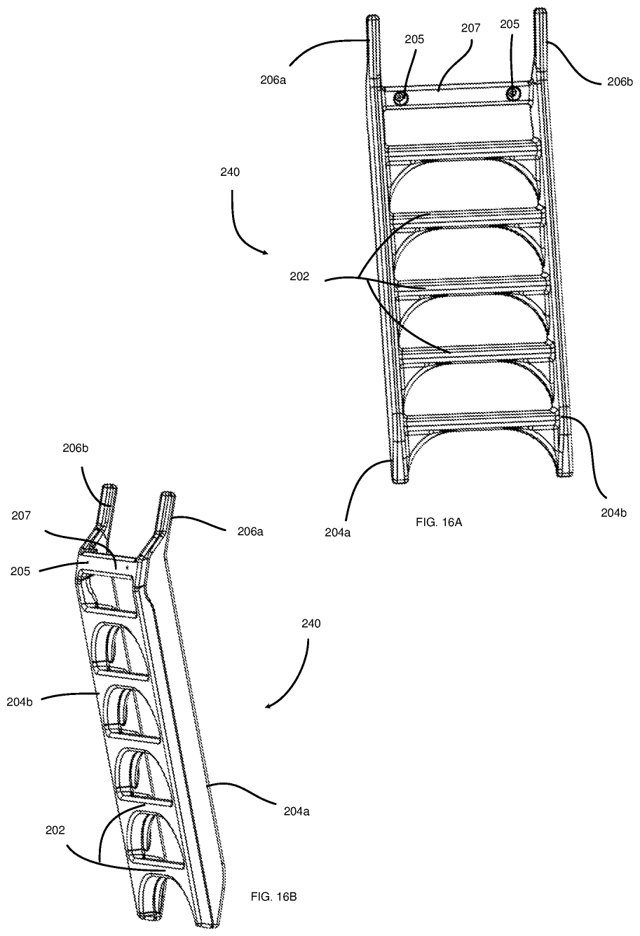

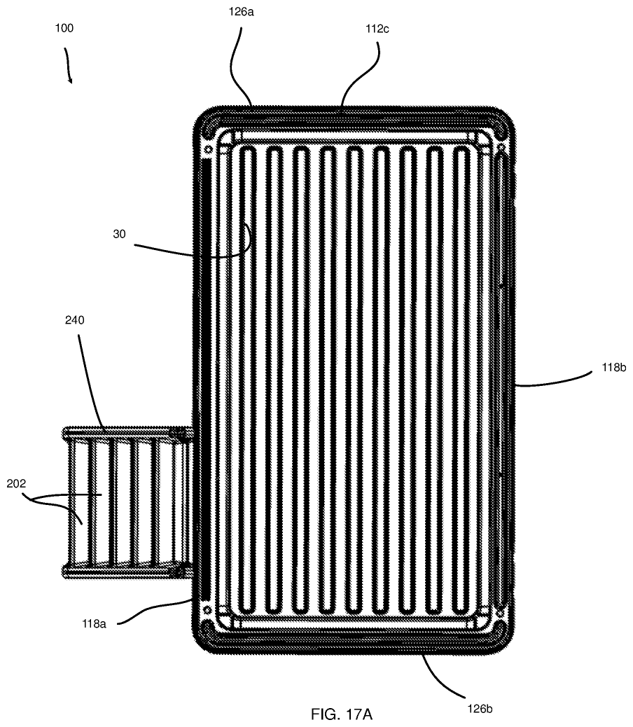

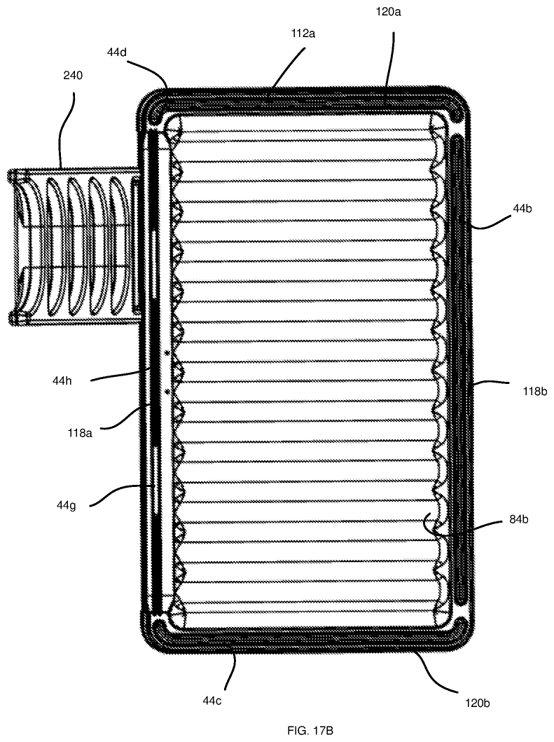

Turning to the drawings provided in FIGS. 1-17B, a sensory enclosure or pod is indicated generally at 10. Sensory enclosure or pod 10 includes a generally horizontal support platform 12 that can rest upon a floor and forms a base of the sensory enclosure 10. The support platform 12 also includes a generally horizontal support surface 14 capable of supporting a mattress and an individual using the sensory enclosure 10. One or more feet 16 can be attached to or formed on a bottom or lower surface 17 of the support platform 12 to raise the support platform 12 above the floor surface.

In one example embodiment of the invention, the one or more feet 16 are elongated such that the sensory enclosure has four or more legs raising the sensory enclosure above the floor surface. The legs can be coupled to the support platform by any number of coupling mechanisms, including threading them into support platform 12, or bolting them to support platform 12. The legs can also comprise leg panels extending a length of sensory enclosure 10. The leg panels can have one or more openings defining steps or rungs that a user or sleeper can use to climb into the sensory enclosure 10.

In one example embodiment of the invention, support platform 12 can also include generally horizontal side support portions 18a and 18b and generally horizontal end support portions 20a and 20b that extend around the support surface 14. The support platform 12 can include a lip 22 that can extend above the support surface 14 and can extend along one or all of the side support portions 18a and 18b and end portions 20a and 20b. Lip 22 is able aids in retaining objects inside of the sensory enclosure 10. Further, lip 22 aids in creating an envelope or zone of comfort within sensory enclosure 10, which is particularly important for individuals with sensory sensitivities.

In another example embodiment, support platform 12 includes one or more drawers built into it to provide storage for clothes and other items. In another example embodiment, a drawer module, platform, or base can be provided that is configured to receive and support the support platform 12. The drawer module or platform includes one or more drawers or doors that are configured to hold a user's clothes and things.

Sensory enclosure 10 also includes one or more generally vertical wall panels that at least partially enclose or extend about a periphery support surface 14. The wall panels create or define a perimeter of the interior space 24 of sensory enclosure 10. As illustrated in FIG. 1, there can be a pair of spaced apart end wall panels 26a and 26b extending upwardly from the end portion 20a and 20b of support platform 12. Sensory enclosure 10 can also include at least one rear or side wall panel 28 that also extends upwardly from the support platform 12 and extends between the end wall panels 26a and 26b.

Sensory enclosure 10 can also include one or more generally horizontal ceiling or roof panels 30 that extend between end wall panels 26a, 26b, and side panel 28. In this embodiment of the invention, an opening 32 opposite side panel 28 is formed by end wall panels 26a, 26b, side wall 28, and ceiling panel 30. The opening 32 allows for easy entry and exit from interior space 24 of sensory enclosure 10.

Some individuals with sensory sensitivities may prefer to have more than one sensory enclosure. For these individuals, the present invention includes one or more generally vertical door panels 40a and 40b that allows an individual the ability to partially or completely close opening 32. The door panels 40a and 40b extend generally between the support platform 12 and the ceiling panel 30. A user can open and close the door panels 40a and 40b by sliding one or more of the panels 40a and/or 40b along a length side support portions 18a or 18b. In another example embodiment of the invention, door panels 40a and 40b can be pivotally coupled to a portion of sensory enclosure 10. In yet another example embodiment of the invention, door panels 40a and 40b can swing or slide from side-to-side or up and down. Other door 40a and 40b configurations are also possible and should be within the spirit and scope of the invention.

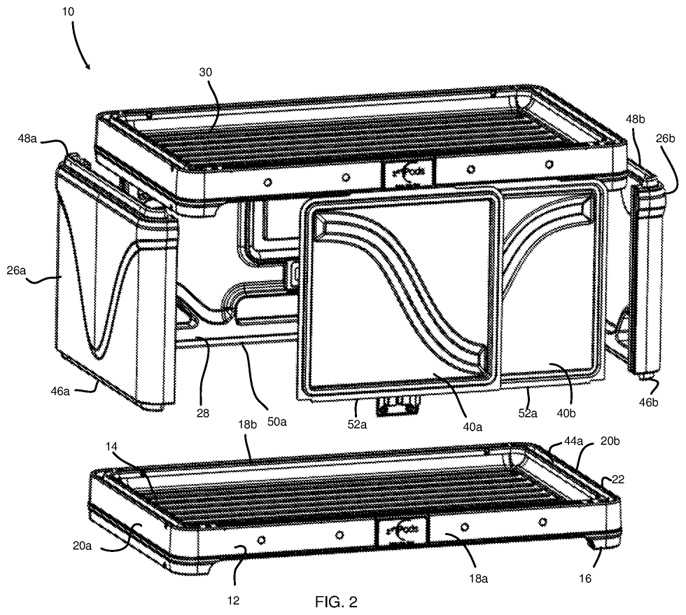

In the embodiment illustrated in FIG. 2, sensory enclosure 10 can be easily assembled and disassembled for movement and different configurations. In this embodiment, lip 22 includes one or more channels 44a extending along its length that have a size and depth to receive a portion of end wall panels 26a and 26b, side wall panel 28, and door panels 40a and 40b. As particularly illustrated in FIG. 2, end wall panels 26a and 26b each have a bottom or lower rail portion 46a and 46b that are sized and shaped to extend into channels 44a extending into lip 22. Door panels 40a and 40b can also have top or upper rail portions 48a and 48b that are sized and shaped to extend into a perimeter channel or groove 44b extending into a portion of ceiling panel 30.

Similar to end wall panels 26a and 26b, side wall panel 28 includes a bottom or lower rail portion 50a and a top or upper rail portion 50b that are also sized and shaped to extend into channels 44a of lip 22 and perimeter channel 40b of ceiling panel 30. Depending upon the configuration of door panels 40a and 40b, they also may include a bottom or lower rail portion 52a and a top or upper rail portion 52b that are also able to extend into channels 44a of lip 22 and perimeter channel 40b of ceiling panel 30. If door panels 40a and 40b are configured to slide alongside support portion 18a, that portion of channel 44a and corresponding portion of channel 44b of ceiling panel 30 may have two or more channels to allow each of door panels 40a and 40b to slide in their own individual channels. In this way, door panels 40a and 40b can slide alongside each other such that in the open position door panels 40a and 40b are next to or parallel to each other and in the closed position they are adjacent to each other extending or spanning opening 32.

At least one of the door panels 40a and 40b can include a safety feature that allows a user to easily separate the door panel 40a or 40b from sensory enclosure 10 in case of an emergency. The safety feature comprises a flex point lip built or formed into lower rail portion 52a and upper rail portion 52b. The flex point comprises a generally narrower section of material that allows the lower rail portion 52a and upper rail portion 52b to bend or flex out of channels of support platform 12. The door panels 40a and 40b can be reinserted after an emergency.

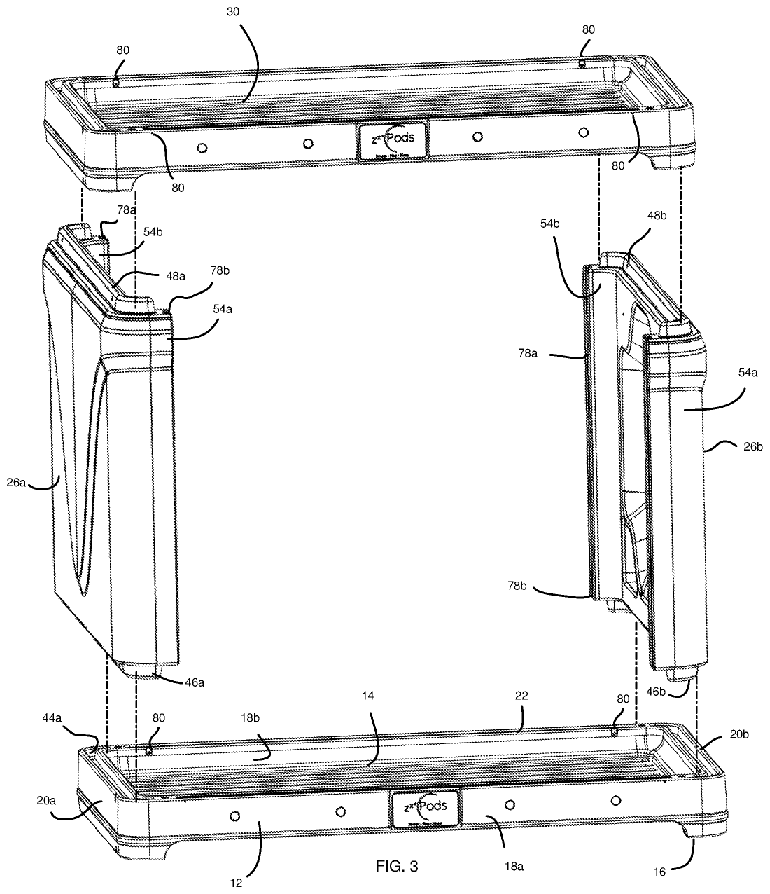

Turning to FIG. 3, positioning or orientation of end wall panels 26a and 26b in relation to support platform 12 and ceiling panel 30 is more clearly illustrated. In this embodiment, end wall panels 26a and 26b are illustrated having curved or rounded corners 54a and 54b, respectively, that wrap around from end support portions 20a and 20b into side support portions 18a and 18b. This configuration and shape of corners 54a and 54b increases the structural support of sensory enclosure 10.

As particularly illustrated in FIG. 3, ceiling panel 30a can comprise another support platform 12. Having ceiling panel 30a be another support platform 12 provides several advantages over the prior art. One advantage includes eliminating an additional piece of manufacturing equipment to form a separate ceiling component of sensory enclosures 10. This reduces the costs involved in manufacturing sensory enclosures 10. Another advantage is the structural support designed into support platform 12 is then used in the ceilings of sensory enclosures 10, which increases the overall structural support and safety of the sensory enclosures 10 of the present invention.

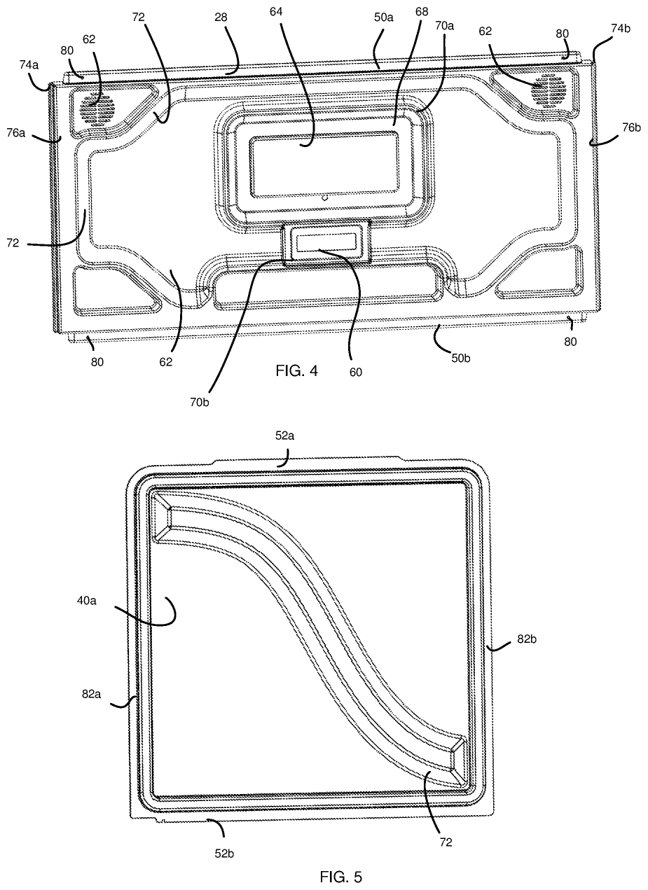

Turning to FIG. 4, side wall panel 28 is illustrated showing unique features of the sensory enclosures 10 of the present invention. These features are particularly important for individuals with sensory sensitivities and sleep problems, especially those with ASD that often deal with sensory issues such as light, sound, colors, etc. One unique is an interior environment control center 60 coupled to one or more speakers 62, a TV or other type of screen 64, lights, fan 66, and the like, all of which allow a user to set, change and control the interior environment of their sensory enclosure 10. While example devices are disclosed, other devices able to customize the interior space 24 and environment are also possible, including but not limited to temperature controls to regulate a desired interior temperature, and tactile or vibration controls to control a vibration of the sensory enclosure. A mirror 68 can also be included in the interior 24 of sensory enclosure 10 to allow a user to view themselves. Other controls are possible and should be considered to be within the spirit and scope of the invention.

In one example embodiment, safety barriers are included to increase the safety of users. For example, some users are very inquisitive and have the ability or desire to disassemble or break objects and devices. Therefore, sensory enclosures 10 of the present include one or more safety barriers. For example, a TV 64 or mirror 68 barrier 70a comprising a see-through sheet or panel is placed over the TV screen 64, and/or mirror 68 to prevent direct access by the user. A control barrier 70b comprising a see-though sheet or panel can be placed over the environment control center 60 to prevent direct access by the user. The barrier 70b can also be tough-sensitive to still allow a user to control the environment control center 60. Fans 66 and speakers 62 can also have barriers such as a grill or enclosure to prevent direct access by the user while allowing air and light to move or pass through the barrier.

Like the structural support of using a support platform 12 for a ceiling panel 30, side wall panel 28 has also been manufactured to further increase the strength of the sensory enclosures 10. As particularly illustrated in FIG. 4, side wall panel 28 is manufactured with one or more ribs or struts 72 extending from its surface. The struts 72 provide rigidity and strength along the length and width of the side wall panel 28. They also provide structural compressive force that can be encountered if weight is placed on ceiling or roof panel 30.

Side wall panel 28 also includes opposed side rails 74a and 74b extending at least partially along a length of each of its end edges 76a and 76b. The opposed side rails 74a and 74b are removably insertable into end channels 78a and 78b formed in the ends of end wall corners 54a and 54b to add additional strength to sensory enclosure 10.

In one example embodiment of the invention, each of rail portions 46a, 46b, 48a, 48b, 50a, 50b, 52a, 52b, and 74a 74b are friction fit into each of their respective channels discussed herein. Alternatively, or in addition to, each of the panels can be coupled, attached, or secured to each other and to support platform 12 and ceiling or roof panel 30. Each of the panels 26a, 26b, side wall panel 28 and support platform 12, and ceiling or roof panel 30 include one or more recesses 80 formed therein to identify fastening or securing locations where fasteners can be inserted to prevent the components or panels of the enclosure or sensory enclosure 10 from separating. Any type of fastener can be used, including screws, bolts, pins, and the like.

Turning to FIG. 5, each of door panels 40a (shown) and 40b (not shown) include door side rails 82a and 82b that are able to fit into or mat with end channels 78b of each of end wall panels 26a and 26b. In this way, door panels 40a and 40b can seal external light from entering the interior space 24 of sensory enclosure 10. Each door panel 40a and 40b can also be formed with a strut 72 extending along its surface to add or provide them with additional structural rigidity. The strut 72 can also have a handle formed into a portion of it to allow a user, especially one with dexterity difficulties, to more easily operate the opening and closing of door panels 40a and 40b.

In one example embodiment, sensory enclosure 10 can include one or more locking mechanisms on the interior 24 or exterior that is able to engage and disengage with a portion of one or more of door panels 26a and 26b to either keep door panels 26a and 26b open, closed, or both. For example, a simple pin mechanism can be inserted into a hole in an end wall panel 26a to engage a part of one or door panels 40a or 40b. In another example embodiment, when door panels 40a and 40 are in an open state and parallel to each other, holes formed each door panel can be aligned allowing for insertion of a pin into the aligned holes, thereby locking them in the open position.

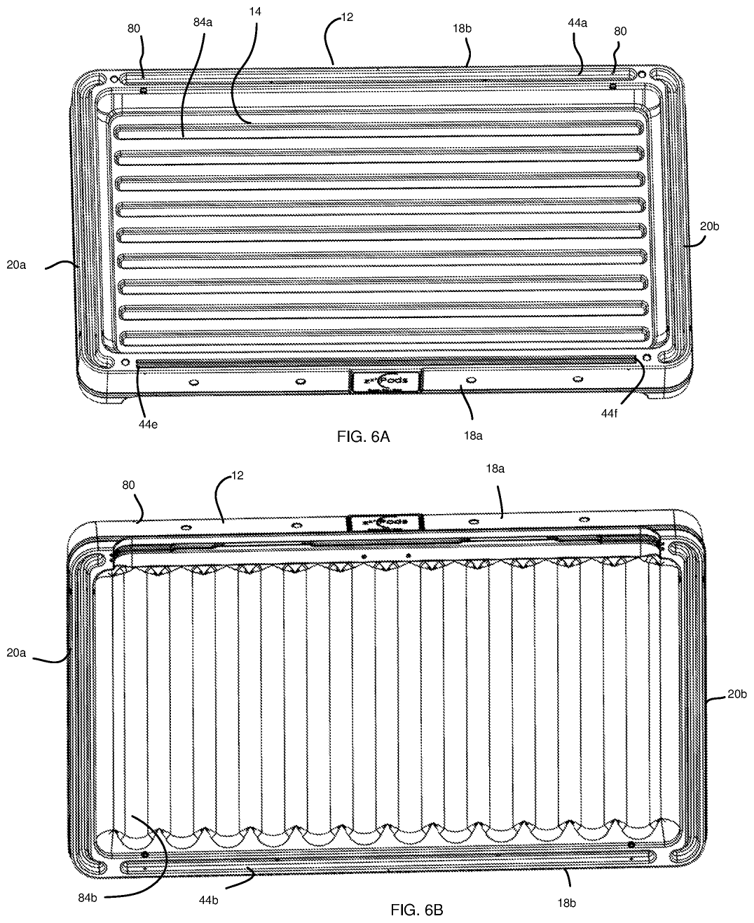

While channel 44a can extend entirely around a perimeter of support platform 12, it can also extend around or along only one or more portions of its perimeter. For instance, in the example illustrated in FIG. 6A, channel 44a includes end channel portions 44c and 44d that extend along end support portions 20a and 20b. The discrete channels 44a, 44c, and 44d allow for a more controlled and precise assembly since each wall panel 26a, 26b, and 28 only fits in its respective channel. As can also be seen in FIG. 6A, one or more door channels 44e and 44f can be formed inside support portion 18a to allow each door panel 40a and 40b to slide along its own channel. Similar door channels 44g and 44h can be formed in a lower surface of support platform 12 or ceiling panel 30.

Support platform 12 can also include one or more elongated grooves 84a formed into support surface 14. The grooves 84a provide a number of functions including creating passages for the movement of air below a mattress. They also act as reservoirs to capture or collect dirt and debris that may accumulate in the interior space 24 during use. The grooves 84a also act as a collection location for liquids such as spilled drinks or accidental nocturnal enuresis. The grooves 84a act to draw any debris or liquids away from any items or furniture inside sensory enclosure 10, for example, a mattress, chairs, bean bags, etc. The grooves 84a allow for easier cleanup by a user, parent, family member, or caregiver. Similar grooves 84b can also be formed on an underside or lower surface of support platform 12 or ceiling panel 30 to permit and channel movement of air through the interior 24 of sensory enclosures 10.

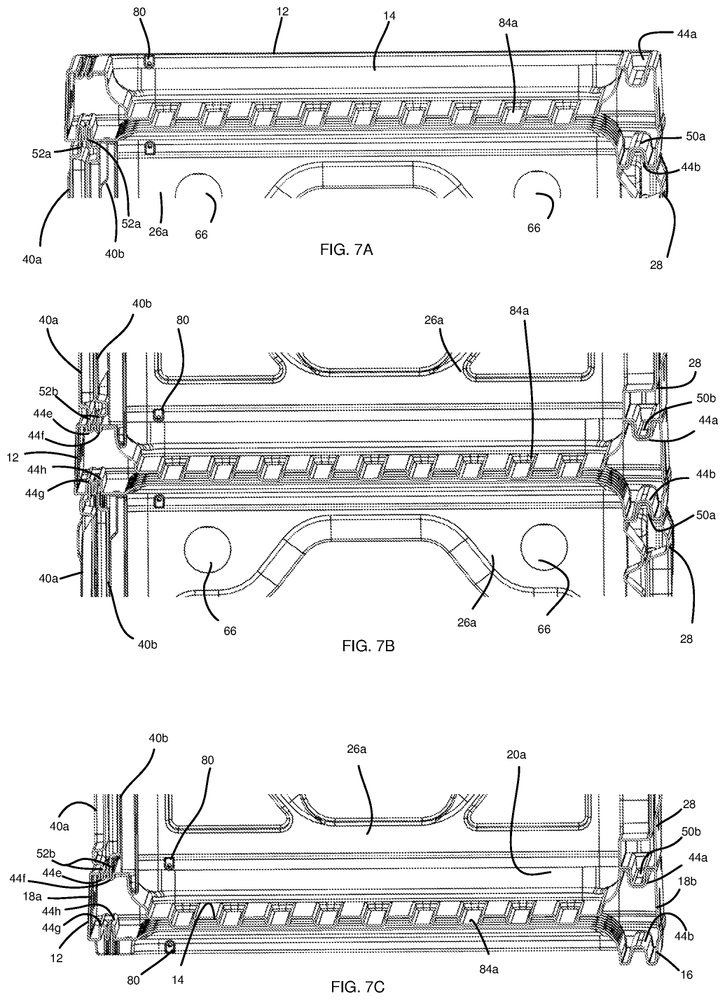

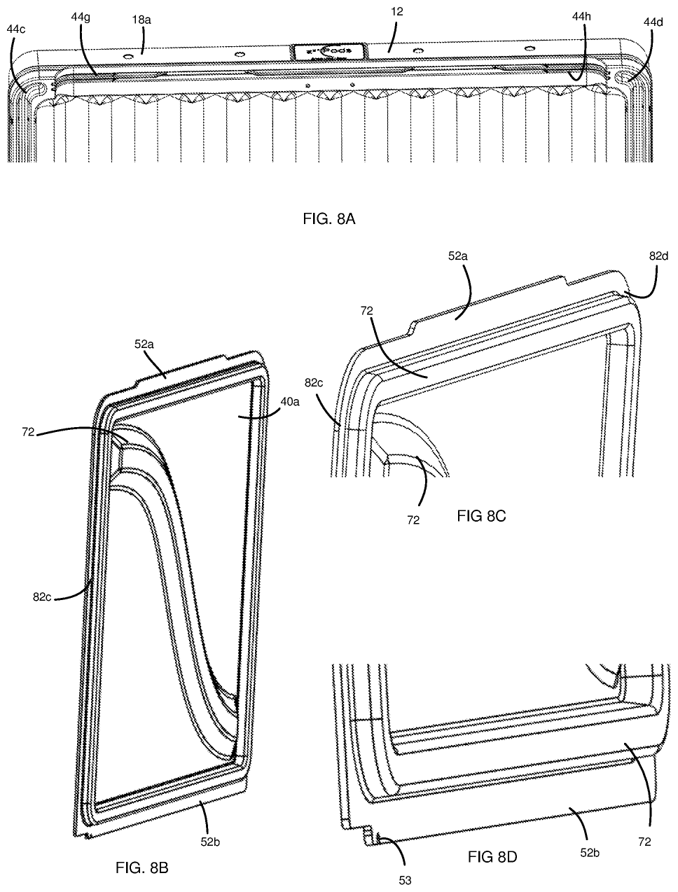

The cross-sectional illustrations of FIGS. 7A, 7B, and 7C depict how each of the components of the present invention are fit together or assembled. For instance, FIG. 7A shows support platform 12 being used as ceiling panel 30. The upper rails 52a and 52b of door panels 40a and 40b are shown positioned in door channels 44g and 44h extending or formed in a lower surface of support platform 12. Similarly, the upper rail portion 50a of side wall panel 28 is shown positioned in channel 44b extending into or formed in a lower surface of support platform 12.

Turning to FIG. 7C, support platform 12 is illustrated assembled with lower door rails 52b of each door panel 40a and 40b positioned in door channels 44e and 44f extending into or formed in support platform 12. The lower side rail 50b of side panel door 28 is positioned in channel 44a extending into or formed in support platform 12. Grooves 84 are also illustrated showing where debris or liquids can be captured and kept a distance away from items within sensory enclosure 10.

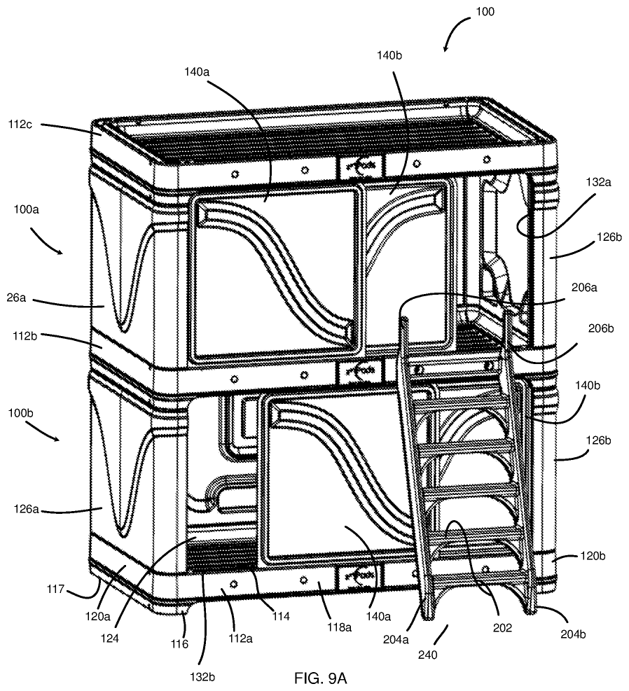

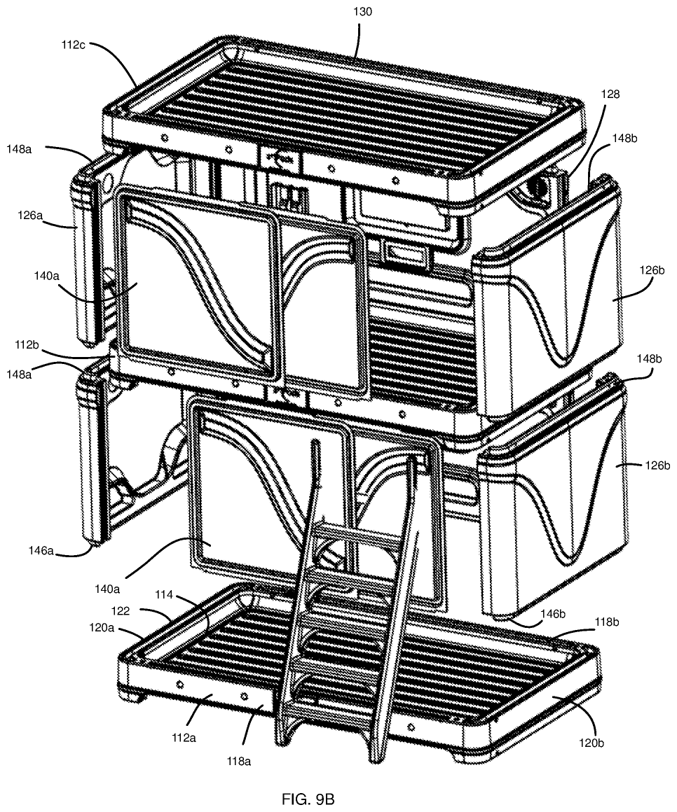

In another example embodiment of the invention, sensory enclosures 10 are capable of being connected in a number of configurations. For example, in FIG. 9A, sensory enclosures 10 can be configured in a stacked or bunked sensory enclosure 100 configuration with one sensory enclosure 100a (upper sensory enclosure) stacked on top of another sensory enclosure 100b (lower sensory enclosure). In this configuration, three support platforms 12 can be used, one as a base support platform 112a positioned on the floor, one as an intermediate support platform 112b that functions as a ceiling or roof for the lower enclosure and a mattress support for the upper enclosure, and a third support platform 112c that functions as a ceiling or roof panel of the upper enclosure.

The stacked sensory enclosure 100 is assembled in the same manner and with the same components as the individual sensory enclosure 10 that was discussed above. For instance, each of the sensory enclosures 100a and 100b includes a generally horizontal support platform 112a and 112b having a support surface 114 capable of supporting furniture, such as a mattress, chair, or bean bag, and an individual using the sensory enclosures 100a and 100b. One or more feet 116 can be attached to or formed on a bottom or lower surface 117 of the support platform 112a to raise the support platform 112a above the floor surface.

Each of the support platforms 112a and 112b can also include generally horizontal side support portions 118a and 118b and generally horizontal end support portions 120a and 120b that extend around the support surface 114. As illustrated in FIG. 9B, each of the support platforms 112a and 112b include a lip 122 that can extend above the support surface 114 and can extend along one or all the side support portions 118a and 118b and end portions 120a and 120b. The lip 122 aids in retaining objects inside of the sensory enclosures 100a and 100b.

The sensory enclosures 100a and 110b each also include one or more generally vertical wall panels that at least partially enclose or extend about a periphery support surface 114. The wall panels of each sensory enclosure 110a and 110b create or define a perimeter of the interior space 24. As with the single sensory enclosure 10, each sensory enclosure 110a and 110b has a pair of spaced apart end wall panels 126a and 126b extending upwardly from the end portion 120a and 120b of support platforms 112a and 112b. Additionally, each sensory enclosure 100a and 100b includes at least one rear or side wall panel 128 that also extends upwardly from the support platforms 112a and 112b and extends between the end wall panels 126a and 126b of each sensory enclosure 100a and 100b.

As briefly mentioned above, the upper sensory enclosure 100a also includes one or more generally horizontal ceiling or roof panels 130 or, as illustrated in FIGS. 9A and 9B, a third support platform 112c. The third support platform 112c or ceiling panel 130 extend between end wall panels 126a, 126b, and side panel 128 of the upper sensory enclosure 100a. Each sensory enclosure 100a and 100b has an opening 132a and 132b, respectively that allows for easy entry and exiting from interior space 124a or 124b of each of the sensory enclosures 100a and 100b. In the stacked or bunked configuration, a user can assemble sensory enclosures 100a and 100b with openings 132a and 132b on the same sides or on opposite sides. With openings 132a and 132b on opposite sides, users of each sensory enclosure 100a and 100b are provided additional privacy.

The sensory enclosures 100a and 100b also include a stair or ladder 200 that allows a user to reach the upper sensory enclosure 100a more easily pod. Stair or ladder 200 can take any number of configurations but generally comprises a number of generally horizontal steps or rungs 202 extending between generally angled ladder rails 204a and 204b. Ladder rails 204a and 204b can be connected or coupled to a portion of sensory enclosures 100a and 100b. In one example embodiments, a portion of stair or ladder 200, such as ladder rails 204a and 204b can be fastened to a portion of one of the support platforms, such as 112b. Ladder 200 is generally placed proximate to opening 132a of upper sensory enclosures 100a. Ladder 200 can take any type of configuration including a straight ladder, curved ladder, spiral ladder, and the like.

Ladder 200 can also include one or more handle portions 206a and 206b that a user can use when using ladder 200. In example embodiment, handle portions 206a and 206b extend beyond an upper end of ladder rails 204a and 204b and can extend a distance that is shorter than or equal to a height of opening 132a. Handle portions 206a and 206b can also include one or more spaced apart holes or openings extending their length to allow a user to wrap their hand completely around a portion of handle portions 206a and 206b. Handle portions 206a and 206b can also include other grasping features such as ridges, knobs, posts, undulations, etc. that can be connected to or formed as part of handle portions 206a and 206b.

Sensory enclosures 100a and 100b can also include one or more interior passages that extend through a portion of support platform 112b. The passage can comprise an opening having a size and shape to allow a user to move between the sensory enclosures 100a and 100b. Stairs or a ladder 200 can be placed in the interior 124 or formed as part of the support platform 112a, 112b, or any of end wall panels and/or side wall panel. In this configuration, upper sensory enclosure 100a can be used as either an upper sensory controlled sleep area or a separate sensory controlled quiet area. The upper sensory enclosure 100a can also be assembled with two side panels 128 rather than door panels to permit access only through the interior passage. It should be appreciated that the components of the present invention permit a user, parent, family member, or caregiver to change any components or panels, or configurations easily and at any time.

Sensory enclosure 10 and sensory enclosures 100a and 100b can also have one or more windows connected to or formed as part of end wall panels, side wall panel, or door panels. Windows can comprise a non-breakable material such as a clear sheet of plastic that allows a user to be able to see through the windows but unable to damage or break them. Having unbreakable windows provides added safety for users.

In another example embodiment of the invention, support platform 12 or 112a can include an inlet and an interior compartment that is designed for holding a weighted material such as water or sand. The weighted material, once added to support platform 12 or 112a helps lower their center of gravity and aids in preventing any accidental movement. This is particularly important for stacked sensory enclosures 100a and 100b.

Sensory enclosure 10 or upper sensory enclosure 110a can also include an upper rail or fence 240 that can be connected or mounted onto and at least partially circling an upper surface of ceiling or roof panel 30 and 130. Rail 240 can have multiple purposes including preventing a user from climbing on top of a sensory enclosure 10 or 100. It can also be added as a safety feature to prevent a user or items placed on the upper surface of ceiling roof panel 30 or 130 from falling off.

In one embodiment of the invention, a mattress can be placed upon the upper surface of ceiling or roof panel 30 to allow sensory enclosure 12 to function as a traditional bunk bed or a sensory controlled quiet area with an upper sleeping area. As illustrated in FIG. 1, rail 240 can be added to prevent a user sleeping in the upper sensory enclosure 100a from rolling off and injuring themselves. In this embodiment, rail 240 can have an opening or break 242 to allow easier access to the upper sensory enclosures 110a. Ladder 200 can also be used by a user to access the upper sensory enclosure 100a.

As illustrated in FIGS. 1 and 12B, rail 240 includes a number of generally vertical posts 244 connected to and positionable proximate to or at corners of ceiling or roof panels 30 and 130. One or more generally horizontal fence rails 246 are connected to or formed as part of posts 244. Fence rails 246 extend between at least two spaced apart posts 244 and run generally along the length and width of a perimeter of ceiling roof panel 30 or 130. Pod rail 240 will generally comprise the same material as sensory enclosures 10 or 100 but can comprise any material, shape, configuration, or design. Rail 240 can also include one or more generally vertical fence rails 247 positioned between posts 244. In another example embodiment, rail 240 comprises fence panels having one or more openings or holes through them. As particularly illustrated in FIG. 1, one or more guides or feet 250 extend generally downward from a bottom or lower surface of pod rail 240 to be guided into and fitted within groove or channel 44b. Once inserted, guides 250 can be secured to the ceiling roof panel 30 with one or more fasteners or friction means.

Sensory enclosures 10 and sensory enclosures 110a and 110b are generally manufactured by a thermoform molding process. The molding process will be discussed in relation to sensory enclosure 10 but it should be understood that sensory enclosures 110a and 110b are stacked sensory enclosures 10 and therefore entail a similar molding process. The molding process begins by selecting an inner sheet and an outer sheet of material (e.g., plastic) for each component (e.g., end wall panel 26a and 26b, side wall panel 28, support platform 12, and ceiling or roof panel 30 (if different from support platform 12)) of sensory enclosure 10. The inner sheets and outer sheets are then molded to the desired shape. Edge or ends of the inner sheets and outer sheets are then connected or assembled to form the component. As illustrated in FIGS. 7A-7C, a space or cavity can be created during the manufacturing process. The cavity is an important aspect of the invention as it allows easy installation and removal of accessories (e.g., TV, speakers, lights, etc.) as wires can be easily run through the cavity.

For any sensory enclosures 10 and 100 having electric capabilities, the cavity allows for mounting of a power control unit in sensory enclosures 10 and 100. The power control unit can connect to an external power source, such as a wall outlet, and to the various electronic components of sensory enclosures 10 and 100. In this way, the power control unit is able to regulate the voltage and currents flowing to the various electronic components. In one embodiment, the power control unit includes a wireless controller (e.g., Bluetooth or Wi-Fi) to allow a user or caregiver to monitor and control the power control unit and/or the individual electronic components.

In another example embodiment, power control unit can also be connected to one or more sensors that are able to monitor an environment inside and/or outside sensory enclosures 10 and 1000. Monitoring the environment is important to ensure that air temperature, air quality, light brightness, sound, etc. are appropriate for the user and for an improvement in their wellbeing and sleeping habits. Other sensors can also be used and should be considered to be within the spirit and scope of the invention.

The sensory enclosures 10 and 100 of the present invention can also include one or more sensory controllers utilizing artificial intelligence (“Sensory AI Controller”) that creates an intelligent and sensory controllable environment that is customizable for individual users. In one example embodiment of the present invention, the Sensory AI Controller comprises a control system and software that is in communication with an array of sensors including, but not limited to, built-in non-invasive sensors, optional wearable sensors, and environmental sensors. The Sensory AI Controller is able to receive, interpret, manage, and control multiple environmental factors (e.g., bed temp, air temp, air circulation, air quality, scent, humidity, light, sound, acoustic vibrations, etc.) from the array of sensors. The Sensory AI Controller is also able to track a user's sensory sensitivity and/or sleep patterns over time and to make automated adjustments to the environment of the sensory enclosure 10 to optimize the user's wellbeing and sleep, and to drive towards improved sensory management and better sleep.

The Sensory AI Controller is also able to receive, monitor, interpret, and track health indicators (e.g., sensory management patterns, amount of time using the sensory enclosure, sleep hours, heart rate, movement, sounds made, etc.) from one or more remote devices (e.g., cellphone, smart watches, etc.). The Sensory AI Controller is able to automatically integrate with a user's health records and data in order to add to their health history, data, and demographics. A user's healthcare provider can be automatically alerted to certain conditions.

Users are able to opt into data sharing for research purposes. Where a user opts in, the Sensory AI Controller can collect and anonymize the user's data. The anonymized data can then be made available in bulk to researchers, who can use the data to solicit subjects for studies and surveys.

In yet another embodiment of the invention, one or more braces 300 can be positioned in the interior or exterior of sensory enclosures 10 or 100. The braces 300 generally comprise a length of material formed into a shape that imparts structural rigidity to it. The braces 300 can include posts, tongues, etc. 302, for inserting into holes or slots in the various components of sensory enclosures 10 or 100. The braces 300 can also include one or more fastening locations 304 such as holes where a fastener such as a bolt can be used to fasten the braces 300 to sensory enclosures 10 or 100. The braces 300 can be placed in a vertical, horizontal, or angular position depending upon the needs of the user. For instance, in the sensory enclosure 100 configuration, a user can place some braces 300 in a vertical position and an angular position in order to distribute the weight of a user on the upper sensory enclosure 110a.

In use, sensory enclosure 10 is assembled by first having an assembler place ground or horizontal support panel or platform 12 on a ground/floor surface (e.g., bedroom floor). In embodiments having feet/legs 16 extending away from horizontal support panel 12, feet/legs 16 are placed against the ground/floor surface. Feet/legs 16 size can be configured to have a size and shape that provides an opening or space between the ground/floor surface and horizontal support panel 12, which provides a grip location for an assembler that is assembling or moving sensory enclosure 10. In one example embodiment of the invention, a logo or other indicia may be provided on a side of horizontal support panel 12 to provide an indicator of the location of an opening 32 or door panel 40a location.

Rear or back panel or wall 28 is then lifted and inserted into or coupled to horizontal support panel 12. In one example embodiment, rear or back panel 28 includes a lower rail 50a that is inserted into channel 44a formed or extending into lip 22 of horizontal support panel 12. Rear or back panel 28 can be secured to horizontal support panel 12 by one or more fasteners extending through a portion of lip 22 and lower rail 50b. One or more caps can be provided to cover the one or more fasteners and/or opening in sensory enclosure 10 that are used to insert the fasteners.

Once rear or back panel or wall 28 is secured in place, assembler then lifts and inserts or couples end panels walls 26a and 26b to horizontal support panel 12. In one example embodiment of the invention, each of end panels 26a and 26b have a lip 52b that is insertable into end channels 44c and 44d extending into a portion of lip 22 in end support portions 20a and 20b of horizontal support panel 12. Once end panels 26a and 26b are mated with horizontal support panel 12 they can be secured in place in a similar fashion as rear panel 28.

An assembler is now able to install ceiling panel 30 by lifting it over vertically or upwardly extending rear panel 28 and end panels 26a and 26b. An assembler guides upper rail 50a of rear panel 28 and upper rails 48a and 48b of end panels 26a and 26b, respectively into groove 44 extending into a lower surface of ceiling panel 30. Ceiling panel 30 is secured in place to rear panel 28 and end panels 26a and 26b by using one or more fasteners in a similar fashion as securing extending through a rear panel 28 and end panels 26a and 26b to horizontal support panel 12.

One or more door panels 40a and 40b may be inserted into opening 32 to allow a user to selective close or block opening 32. An assembler guides upper rail 52a of one of door panels 40a and 40b into a first groove or track 44f in ceiling panel 30 and then guides its lower rail 52b into a first groove or track 44f in horizontal support panel or platform 12. Assembler repeats the process with the subsequent door by installing upper rail 52a and lower rail 52b in a second groove or track 44e of horizontal support panel 12 and ceiling panel 30. Assembler is then able to connect power to control center 60, which supplies power to all of the sensory accessories of sensory enclosure 10.

Assembler is able to place bedding or a mattress into sensory enclosure 10 at various points during assembly. For example, the assembler is able to position the bedding on support panel 12 prior to assembly of any of the other panels. An assembler may also wait to position the bedding until all but the ceiling panel 20 or door panels 40a and 40b are installed. While use of bedding has been described herein, it should also be appreciated that sensory enclosure 10 can be used without bedding.

In another example embodiment of the invention, an assembler is able to secure one or more fence rails 240 to an upper surface of ceiling panel 30. In this manner, fence rails 240 extend about all or a portion of a perimeter of ceiling panel 30. The area enclosed by fence rails 240 may be used for storage. In another example embodiment, fence rails 240 define an additional sleeping or play area and act to prevent accidental falls from sensory enclosure 10. A ladder 200 may be provided to allow a user to access the fenced area. Ladder 200 can be secured to a portion of sensory enclosure 10 to prevent its accidental separation therefrom.

In yet another example embodiment of the invention, multiple sensory enclosures 10 may be connected or stacked together in either a side-by-side configuration or a bunkbed configuration.

It will be readily apparent to those of ordinary skill in the art that many modifications and equivalent arrangements can be made thereof without departing from the spirit and scope of the present disclosure, such scope to be accorded the broadest interpretation of the appended claims to encompass all equivalent structures and products. Moreover, features or aspects of various example embodiments may be mixed and matched (even if such combination is not explicitly described herein) without departing from the scope of the invention.

What is claimed is:

1. A sensory enclosure assembly having an interior compartment conducive to sensory or sleep management, comprising:
a support platform having a support surface configured to support a weight;
one or more wall panels extending upwardly from the support platform and at least partially surrounding the support surface;
a ceiling panel resting upon and extending between the one or more wall panels; and
at least one sensory accessory disposed in the interior compartment;
wherein the at least one sensory accessory is adjustable to control an environment of the interior compartment.

2. The sensory enclosure assembly of claim 1, further comprising one or more door panels operatively extending between the support platform and the ceiling platform to permit egress and ingress of the interior compartment.

3. The sensory enclosure assembly of claim 1, further comprising a control center operatively coupled to the at least one sensory accessory to control adjustment of the environment of the interior compartment.

4. The sensory enclosure assembly of claim 1, wherein the at least one sensory accessory comprises one or more lights, thermostats, speakers, scent emitters, sound generators, vibration generators, headphones, and virtual reality devices, or combinations thereof.

5. The sensory enclosure assembly of claim 4, wherein the control center comprises a user interface positioned in the interior compartment that is selectable by a user wanting to control the one or more sensory accessory.

6. The sensory enclosure assembly of claim 4, wherein the control center comprises a computer interface in operative communication with the interior compartment and capable of controlling the one or more sensory accessory.

7. The sensory enclosure assembly of claim 6, wherein the computer interface comprises artificial intelligence configured to control the one or more sensory accessory to control the sensory environment of the interior compartment for a user having sensory needs.

8. The sensory enclosure assembly of claim 1, further comprising a mirror in the interior compartment.

9. The sensory enclosure assembly of claim 1, further comprising at least two sensory enclosures stacked in a vertical configuration.

10. The sensory enclosure assembly of claim 9, further comprising a passageway extending between the at least two sensory enclosures stacked in the vertical configuration.

11. A method of managing a sensory or sleep environment, comprising:
providing a sensory enclosure having an interior compartment, the sensory enclosure comprising:
a support platform having a support surface configured to a weight;
one or more wall panels extending upwardly from the support platform and at least partially surrounding the support surface;
a ceiling panel resting upon and extending between the one or more wall panels; and
providing at least one sensory accessory disposed in the interior compartment; and
providing a control center operatively coupled to the at least one sensory accessory to control adjustment of an environment of the interior compartment;
wherein the control center is operatively controlled by a user or a computer interface.

12. The method of managing a sensory or sleep environment of claim 11, further comprises providing one or more door panels operatively extending between the support platform and the ceiling platform to permit egress and ingress of the interior compartment.

13. The method of managing a sensory or sleep environment of claim 11, wherein the at least one sensory accessory comprises one or more lights, thermostats, speakers, scent emitters, sound generators, vibration generators, headphones, and virtual reality devices, or combinations thereof.

14. The method of managing a sensory or sleep environment of claim 11, wherein the control center comprises a user interface positioned in the interior compartment that is selectable by a user wanting to control the one or more sensory accessory.

15. The method of managing a sensory or sleep environment of claim 13, wherein the computer interface comprises artificial intelligence configured to control the one or more sensory accessory to control the sensory environment of the interior compartment for a user having sensory needs.

16. The method of managing a sensory or sleep environment of claim 11, further comprises providing a mirror in the interior compartment.

17. The method of managing a sensory or sleep environment of claim 11, further comprises providing at least two sensory enclosures stacked in a vertical configuration.

18. The method of managing a sensory or sleep environment of claim 17, further comprises providing a passageway extending between the at least two sensory enclosures stacked in the vertical configuration.

19. A sensory enclosure system having one or more interior compartments that are conducive to improving sensory management or sleeping habits, comprising:
a base support platform having a base support surface configured to support a weight;
one or more base wall panels resting upon the base support surface of the base support platform and at least partially surrounding the base support surface;
a secondary support platform having a secondary support surface configured to support a mattress, wherein the secondary support platform rests upon and extends between the one or more base wall panels;
one or more secondary wall panels resting upon the secondary support surface of the secondary support platform and at least partially surrounding the secondary support surface;
a ceiling panel resting upon and extending between the one or more secondary wall panels;
at least one sensory accessory comprises one or more lights, thermostats, speakers, scent emitters, sound generators, vibration generators, headphones, and virtual reality devices, or combinations thereof; and
one or more door panels operatively resting upon and extending between the base support platform and the secondary support platform to permit egress and ingress into the interior.

20. The sensory enclosure system of claim 20, further comprising a control center comprises a user interface positioned in the interior compartment that is selectable by a user wanting to control the one or more sensory accessory.
